(19) United States
(12) Patent Application Publication
(10) Pub. No.: US 2020/0121561 A1
(43) Pub. Date: Apr. 23, 2020

(54) EMERGENCY MEDICATION STORAGE DEVICE FOR CARDIAC CHEST PAIN

(71) Applicant: SmHeartCard Inc., Edmonton (CA)

(72) Inventors: John R. MACKEY, Edmonton (CA); James STEWART, Edmonton (CA)

(21) Appl. No.: 16/605,963

(22) PCT Filed: Apr. 9, 2019
(86) PCT No.: PCT/CA2019/050428

§ 371 Date: Oct. 17, 2019

Related U.S. Application Data
(60) Provisional application No. 62/655,382, filed on Apr. 10, 2018.

(51) Int. Cl. A61J 7/00; A61J 1/03; A61K 31/21; A61K 31/616; A61P 9/10; A45C 11/00; B65D 77/24

(52) U.S. Cl. CPC A61J 7/0084; A61J 1/03; A61K 31/21; A45C 2011/007; A61P 9/10; A45C 11/00; B65D 77/24; A61K 31/616

(57) ABSTRACT

A packaging system for emergency access to cardiac medications, where the patient has convenient and safe access to aspirin and nitroglycerin for immediate use at the onset of cardiac chest pain or suspected cardiac chest pain.

### CROSS REFERENCE TO RELATED APPLICATION

This application claims priority to U.S. provisional patent application 62/655,382, filed Apr. 10, 2018, the entire contents of which is hereby incorporated by reference.

### FIELD

The present invention discloses an emergency cardiac medication storage device.

### BACKGROUND

Patients with known or suspected coronary artery disease (CAD) are instructed to have immediate access to acetylsalicylic acid (ASA; also known as aspirin) and to nitroglycerin (NTG). Standard medical recommendations for people with known or suspected CAD who develop chest pain are to immediately chew and swallow a 325 mg dose of ASA, and to take a single dose of a nitroglycerin preparation (Reference: O'Gara et. al Journal of the American College of Cardiology, Vol. 61, No. 4, e78-140, 2013. 2013 ACCF/AHA Guideline). If pain is not relieved, a second dose of nitroglycerin is to be taken five minutes later. If pain persists, a third dose of nitroglycerin is to be taken five minutes later. If pain persists after three doses of NTG, emergency medical care is required.

Nitroglycerin (NTG), also known as nitroglycerine, trinitroglycerin (TNG), trinitroglycerine, nitro, glyceryl trinitrate (GTN), or 1,2,3-trinitroxypropane, is a heavy, colorless, oily, explosive liquid. It is used to treat and prevent chest pain from not enough blood flow to the heart (angina) or and this includes chest pain from a heart attack. It is taken by mouth, under the tongue, applied to the buccal mucosa, applied to the skin, or by injection into a vein.

NTG has physical properties that complicate its storage. NTG is volatile. NTG can react with atmospheric oxygen. NTG is light sensitive and degrades with light exposure. NTG adsorbs to most plastics commonly used to store and distribute tablets. NTG adsorbs to cotton used to hold pills in pill bottles.

Patients with known or suspected coronary artery disease are typically prescribed a preparation of nitroglycerin (NTG) and instructed to keep it on hand to be used at the onset of chest pain. Compliance with these recommendations is very poor. In a case series of 38 consecutive patients with CAD attending a primary care setting, only 7 of 38 (18%) prescribed NTG were carrying it. (Reference: C. Horsman and T. Frederick. Poor compliance with carrying nitroglycerin and aspirin in patients with coronary artery disease. Canadian Journal of Cardiology, (2018) doi: 10.1016/ j.cjca.2018.03.005)

Time to treatment with ASA and NTG after myocardial infarction is frequently delayed. In one quality assurance survey of a Canadian metropolitan hospital, median time to ASA treatment was 90 minutes after presenting to emergency room with myocardial infarction (Underutilization of acetylsalicylic acid for acute coronary syndromes in the emergency department. Journal of the Canadian Association of Emergency Physicians. Dufresne F. et. al. 2004).

### SUMMARY

In one aspect there is described a medicament holder, comprising:
a base (5) having a surface, the surface comprising a first medicament holder receptacle (5a) and a second medicament holder receptacle (5b);
a first insert holder (8) removably disposed with the first medicament holder receptacle (5a);
a second medicament insert (9) removably disposed with the second medicament holder (5b), the second medicament insert (9) is configured to removably store at least one second medicament (10);
a first medicament insert (4) removably disposed with the first insert holder (8), the first medicament insert (4) is configured to removably store at least one first medicament (3);
a first cap liner (2) removably disposed on the first insert holder (8);
a second cap liner (11) removably disposed on the second medicament insert (9); and
a cap (14) comprising a first cap element (1), a second cap element (12), and a bridge (13) connecting said first element (1) and said second cap element (12), the first cap element (1) is configured for removable attachment to said first medicament holder receptacle (7a) and the second cap element (13) is configured for removable attachment to said second medicament holder receptacle (7b).

In one example, said base (7) comprises polypropylene, nylon, plastics, metals, or combinations thereof.

In one example, said first holder insert holder (8) comprises PTFE.

In one example, said first cap liner (2) comprises PTFE, NXT 85 PTFE, fluoropolymer, silicone rubber, or plastics.

In one example, further comprising a layer of aluminum.

In one example, said second cap liner (11) comprises plastic, silicon rubber, paper, or combinations thereof.

In one example, said second medicament holder (9) comprises polypropylene.

In one example, the first medicament holder (4) comprises PTFE, NTX 85 PTFE, glass, boroscilicate glass, brown borosilicate glass, Macor, or the like.

In one example, said first cap element (1) and said second cap element (12) comprise polypropylene, nylon, plastics, metals, or combinations thereof.

In one example, further comprising at least one first medicament (3) disposed within at least one aperture within said first medicament insert (4).

In one example, said at least one first medicament (3) is nitroglycerin (NTG).

In one example, further comprising three first medicaments (3) disposed in each of three apertures within said first medicament insert (4).

In one example, each said first medicament (3) comprises or consists of nitroglycerin (NTG).

In one example, said NTG comprises granules, a tablet(s), or oral fast dissolving film.

In one example, the oxygen within the aperture of the first medicament insert (4) is reduced by dilution with nitrogen gas or carbon dioxide.

In one example, further comprising at least one second medicament (10) disposed within at least one aperture with said second insert holder (9).

In one example, said second medicament (10) comprises or consists of acetylsalicylic acid (ASA).

In one example, further comprising four second medicaments (10) disposed in each of four aperture within said second medicament holder (9).

In one example, said second medicament (10) comprises or consists of acetylsalicylic acid (ASA).

In one example, each said ASA is 81 mg ASA.

In one example, further comprising instructions for use (7) on the surface of the base (5).

In one example, further comprising tamper-resistant tape placed the cap (14).

In one aspect there is described a method of storing nitroglycerin (NTG) and acetylsalicylic acid (ASA) for use in the treatment of known or suspect coronary artery disease (CAD), comprising: providing a medicament holder of any one of claims 1 to 21, said medicament holder comprising NTG and ASA.

In one aspect there is described a method of treating a human subject having or suspected of having coronary artery disease (CAD), comprising administering nitroglycerin (NTG) and acetylsalicylic acid (ASA) obtained from the medicament holder of any one of claims 1 to 21, said medicament holder comprising NTG and ASA.

In one aspect there is described a use of nitroglycerin (NTG) and acetylsalicylic acid (ASA) obtained from the medicament holder of any one of claims 1 to 21, said medicament holder comprising NTG and ASA for treating a human subject having or suspected of having coronary artery disease (CAD).

In one aspect there is described a kit for a medicament holder, comprising:
a base (5) having a surface, the surface comprising a first medicament holder receptacle (5a) and a second medicament holder receptacle (5b);
a first insert holder (8) removably disposed with the first medicament holder receptacle (5a);
a second medicament insert (9) removably disposed with the second medicament holder (5b), the second medicament insert (9) is configured to removably store at least one second medicament (10);
a first medicament insert (4) removably disposed with the first insert holder (8), the first medicament insert (4) is configured to removably store at least one first medicament (3);
a first cap liner (2) removably disposed on the first insert holder (8);
a second cap liner (11) removably disposed on the second insert holder (9);
a cap (14) comprising a first cap element (1), a second cap element (12), and a bridge (13) connecting said first element (1) and said second cap element (12), the first cap element (1) is configured for removable attachment to said first medicament holder receptacle (7a) and the second cap element (13) is configured for removable attachment to said second medicament holder receptacle (7b).

In one example, said base (7) comprises polypropylene, nylon, plastics, metals, or combinations thereof.

In one example, said first holder insert holder (8) comprises PTFE.

In one example, said first cap liner (2) comprises PTFE, NXT 85 PTFE, fluoropolymer, silicone rubber, or plastics.

In one example, further comprising a layer of aluminum.

In one example, said second cap liner (11) comprises plastic, silicon rubber, paper, or combinations thereof.

In one example, said second insert holder (9) comprises polypropylene.

In one example, the first medicament holder (4) comprises PTFE, NTX 85 PTFE, glass, boroscilicate glass, brown borosilicate glass, Macor, or the like.

In one example, said first cap element (1) and said second cap element (12) comprise polypropylene, nylon, plastics, metals, or combinations thereof.

In one example, further comprising at least one first medicament (3) disposed within at least one aperture within said first medicament insert (4).

In one example, said at least one first medicament (3) is nitroglycerin (NTG).

In one example, further comprising three first medicaments (3) disposed in each of three apertures within said first medicament insert (4).

In one example, each said first medicament (3) comprises or consists of nitroglycerin (NTG).

In one example, said NTG comprises granules, a tablet(s), or oral fast dissolving film.

In one example, the oxygen within the aperture of the first medicament insert (4) is reduced by dilution with nitrogen gas or carbon dioxide.

In one example, further comprising at least one second medicament (10) disposed within at least one aperture with said second insert holder (9).

In one example, said second medicament (10) comprises or consists of acetylsalicylic acid (ASA).

In one example, further comprising four second medicaments (10) disposed in each of four aperture within said second medicament insert (9).

In one example, said second medicament (10) comprises or consists of acetylsalicylic acid (ASA).

In one example, each said ASA is 81 mg ASA.

In one example, further comprising instructions for use (7) on the surface of the base (5).

In one example, further comprising tamper-resistant tape placed the cap (14).

### BRIEF DESCRIPTION OF THE FIGURES

Embodiments of the present disclosure will now be described, by way of example only, with reference to the attached FIGS. 1, and 2.

FIG. 1. The cardiac medication holder disassembled. This demonstrates: 1) the nitroglycerin cap; 2) the NTG cap liner; 3) three NTG tablets; 4) the NTG insert—this disc has three individual wells to hold three individual doses of NTG, minimizing the atmospheric exposure and physical mobility of the NTG pills, and surrounding the NTG doses with chemically inert material; 5) the base; 6) grooves—to facilitate placement of tamper-resistant tape; 7) instructions—words on the card describe how to take ASA and NTG; 8) the NTG insert holder; 9) the ASA well; 10) ASA tablets; 11) the ASA cap liner; 12) the ASA cap; 13) the cap bridge—this facilitates proper orientation of the NTG and ASA cap.

FIG. 2. The cardiac medication holder fully assembled. This demonstrates: 1) the nitroglycerin cap; 5) the base; 6) grooves—to facilitate placement of tamper-resistant tape; 7) instructions—words describing how to take ASA and NTG; 12) the ASA cap; 13) the cap bridge—this facilitates proper orientation of the NTG and ASA cap.

FIG. 3 depicts on example of an embodiment of the present application.

FIG. 4 depicts release test work flow.

FIG. 5 depicts Minimum and maximum pocket temperature measured with ThermPro-TP50 Digital LCD Indoor Thermometer Hygrometer Humidity Meter.

FIG. 6 depicts Heat flow of Nitrostat® at various temperatures for 1 week from the microcalorimeter.

FIG. 7 depicts Release profiles of Nitrostat® at various temperatures from the microcalorimeter. (n=6 mean±SEM).

FIG. 8 depicts Release profiles of Nitrostat® in original glass packaging, emergency cardiac medication holder enclosed with Polytetrafluoroethylene (PTFE) or Polyethelene (PE) at room temperature (22° C.), or 35° C. at week 1 (a), week 4 (b), and week 12 (c). (n=6 mean±SEM).

### DETAILED DESCRIPTION

Generally, the present invention relates to a cardiac medication holder specifically designed to stabilize NTG and ASA tablets to be carried on a person, including in a shirt pocket, pant pocket, purse or wallet. The design enables users to keep these medications discretely and conveniently available, and minimizes the degradation of NTG that would otherwise occur due to oxidation, light exposure, adsorption to plastic, and degradation augmented by extreme temperature and humidity variation.

One contribution to the lack of NTG availability in persons with known or suspected CAD is that the existing preparations of NTG are not convenient to carry on a person. In the Horsman case series patients complained that the prescribed nitroglycerin preparations were inconvenient to carry; males in particular found sublingual nitroglycerin spray preparations to be inconvenient. Only two of 17 male CAD patients (12%) were carrying the prescribed NTG preparation. (Reference: C. Horsman and T. Frederick. Poor compliance with carrying nitroglycerin and aspirin in patients with coronary artery disease. Canadian Journal of Cardiology, (2018) doi: 10.1016/ j.cjca.2018.03.005). One contribution to the lack of NTG availability in persons with known or suspected CAD is that existing preparations of NTG are not discrete. One of the common NTG preparations prescribed is a sublingual spray bottle; patients describe this bottle as large, bulky, and inconvenient to carry, and noticeable in pant pockets or shirt pockets. Sublingual and buccal tablets of NTG are generally kept in sealed glass bottles because the sublingual and buccal NTG preparations are volatile, and adsorb to most of the plastics commonly used to store and handle tablets. Sublingual and buccal tablets of NTG are generally kept in opaque containers to reduce degradation by light exposure, and most plastics used in bubble preparations are transparent and contribute to light induced degradation. Sublingual and buccal tablets of NTG are generally stored in air-tight containers to reduce exposure to oxygen and to reduce the escape of the volatile NTG. Sublingual and buccal tablets of NTG should not be packed in bottles with cotton because cotton adsorbs NTG. Repackaging in standard bubble pack preparations would be expected to increase this oxygen exposure due to the relatively large volume of free air surrounding a small tablet. Repackaging NTG in standard bubble pack preparations would be expected to reduce NTG content of the tablet due to adsorption by the plastics used in bubble packs.

Polytetrafluoroethylene (PTFE) is a synthetic fluoropolymer of tetrafluoroethylene that has numerous applications. The best known brand name of PTFE-based formulas is Teflon by Chemours. Chemours is a 2015 spin-off of DuPont Co., which discovered the compound in 1938. Due to the extremely strong carbon-fluorene bonds, PTFE is extremely inert. Nitroglycerin does not interact with PTFE. PTFE, unlike most other plastics, does not adsorb NTG.

PTFE is one of a class of inert plastics called fluoropolymers, which includes PVF (polyvinylfluoride); PVDF (polyvinylidene fluoride); PTFE (polytetrafluoroethylene); PCTFE (polychlorotrifluoroethylene); PFA (perfluoroalkoxy polymer); FEP (fluorinated ethylene-propylene); ETFE (polyethylenetetrafluoroethylene); ECTFE (polyethylenechlorotrifluoroethylene); FFPM/FFKM (Perfluorinated Elastomer [Perfluoroelastomer]); FPM/FKM (Fluorocarbon [Chlorotrifluoroethylenevinylidene fluoride]); FEPM (Fluoroelastomer [Tetrafluoroethylene-Propylene]); PFPE (Perfluoropolyether); and PFSA (Perfluorosulfonic acid).

One commercially available form of PTFE is NXT 85 that has decreased porosity when compared to standard production methods of PTFE.

Borosilicate glass is a type of glass with silica and boron trioxide as the main glass-forming constituents. Borosilicate glasses are known for having very low coefficients of thermal expansion making them resistant to thermal shock. Such glass is commonly used for the construction of reagent bottles. Borosilicate glass is sold under such trade names as Borcam, Borosil, DURAN, Suprax, Simax, BSA 60, BSC 51, Heatex, Endural, Schott, Refmex, Kimble, and Pyrex.

Macor is the trademark for a machineable glass-ceramic developed and sold by Corning Inc. It is a white material that looks somewhat like porcelain. Macor is a good thermal insulator and is stable up to temperatures of 1000° C., with very little thermal expansion or outgassing. It can be machined using standard metalworking tools.

By volume, dry air contains 78% nitrogen gas, 21% oxygen, 0.93% argon, 0.04% carbon dioxide, and small amounts of other gases. Diluting room air with nitrogen gas reduces its oxygen content, and will reduce the rate of NTG degradation by the process of oxydation. N2 gas is heavier than atmospheric air and settles into the lowest available layer. Diluting room air with carbon dioxide gas will reduce its oxygen content, and will reduce the rate of NTG degradation by oxidation. CO2 gas is heavier than atmospheric air and settles into the lowest available layer.

Acetylsalicylic acid (ASA), also known as Aspirin, is a medication used to treat pain, fever, or inflammation. ASA given shortly after a heart attack decreases the risk of death. ASA does not react with common plastics. ASA requires protection from high humidity environments. Many persons with known or suspected CAD do not carry this drug on their person for emergency access with the onset of chest pain, despite recommendations to carry ASA. (Reference: O'Gara et. al Journal of the American College of Cardiology, Vol. 61, No. 4, e78-140, 2013. 2013 ACCF/AHA Guideline). In a case series of 38 CAD patients, none carried ASA. (Reference: C. Horsman and T. Frederick. Poor compliance with carrying nitroglycerin and aspirin in patients with coronary artery disease. Canadian Journal of Cardiology, (2018) doi: 10.1016/ j.cjca.2018.03.005).

Temperature variation in personal carrying locations such as wallets and pockets exceeds the standard temperature recommendations for stability of NTG, which is 15 to 30 degrees Celsius. We have found temperatures range from 12 to 32 degrees Celsius, depending on location of storage, physical activity of the individual, clothing choices, and weather conditions. (Mackey et. al. unpublished data). Consequently, measures to improve NTG stability are required to make it more suitable for personal carrying.

Patients with ongoing chest pain may not be able to recall instructions on how to use NTG or take ASA pills, and may not be able to readily read instructions on pill bottles. Instructions for use of these medications on the device would improve correct use of these medications.

The following sets out a broad description of the many different embodiments of the present invention. This description is only an example and does not describe every possible permutation of the features and composition of the product.

The term “well” refers to an enclosure formed from a solid material that has the appropriate properties to store NTG and/or ASA. The surface material of the well is in direct contact with the medication.

The term “insert” refers to a specific component made from a material other than the base material of the card. It fits into, and is supported physically by, the base.

The term “base” refers to the majority of the substance of the cardiac medication holder, and is the components into which the insert is seated.

The term “cap liner” refers to the disc that is seated within the cap, and presents the face towards the interior of the well. The inner surface of the cap liner is exposed to the medication.

The term “cap” refers to the protective lid or cover that holds the cap liner and thereby protects the medications from exposure to light and the atmosphere.

The term “syringe” refers to a tube with a nozzle and piston or bulb for sucking in and ejecting fluids. The term fluid includes gases.

The “base” can be made of a variety of materials, including without limitation polypropylene, nylon, other plastics, metals, or combinations thereof.

The “cap” can be made of a variety of materials, including without limitation polypropylene, nylon, other plastics, metals, or combinations thereof.

The “cap liner” exposed to NTG can be made of a variety of materials, including PTFE, NXT 85 PTFE, other fluoropolymers, silicone rubber, or other plastics. The cap liner may or may not have a layer of aluminum to reduce permeability.

The “cap liner” exposed to ASA can be made of a variety of materials, including a variety of plastics, silicone rubber, paper, and combinations thereof.

The “NTG insert” can be made of a variety of materials, including PTFE, NTX 85 PTFE, glass, borosilicate glass, brown borosilicate glass, Macor, or other materials with the appropriate chemical characteristics suitable for exposure to and stabilization of NTG.

This invention is conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

The embodiments described herein are intended to be examples only. Those of skill in the art can effect alterations, modifications and variations to the particular embodiments. The scope of the claims should not be limited by the particular embodiments set forth herein, but should be construed in a manner consistent with the specification as a whole.

The following is a general description of the use and utility of this invention.

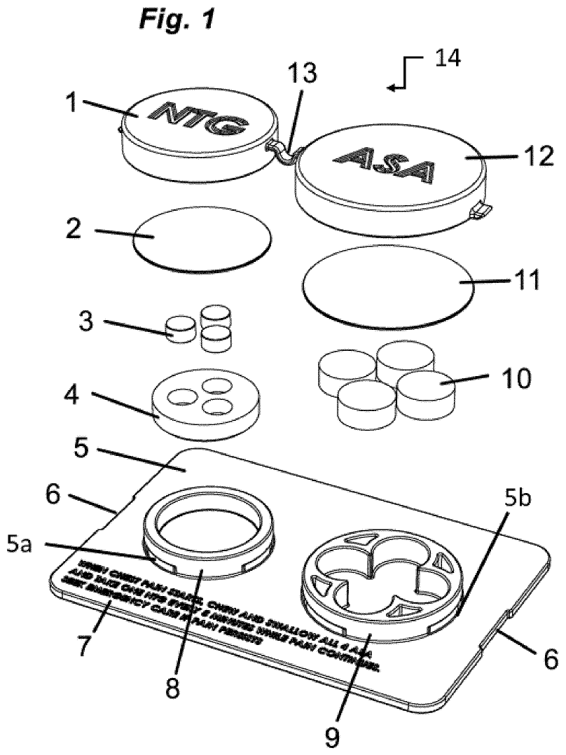

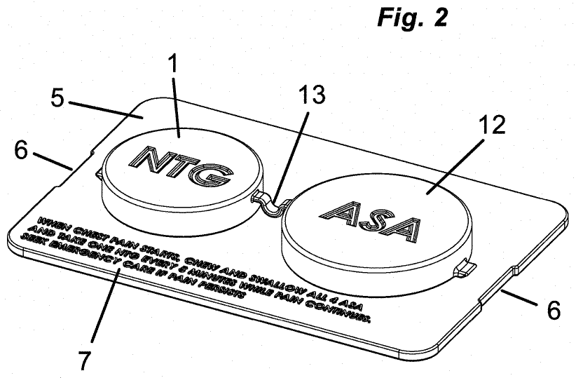

As depicted in FIGS. 1 and 2, in one aspect, there is provided a medicament holder, comprising: a base (5) having a surface, the surface comprising a first medicament holder receptacle (5a) and a second medicament holder receptacle (5b); a first insert holder (8) removably disposed with the first medicament holder receptacle (5a); a second medicament insert (9) removably disposed with the second medicament holder (5b), the second medicament insert (9) is configured to removably store at least one second medicament (10); a first medicament insert (4) removably disposed with the first insert holder (8), the first medicament insert (4) is configured to removably store at least one first medicament (3); a first cap liner (2) removably disposed on the first insert holder (8); a second cap liner (11) removably disposed on the second insert holder (9); a cap (14) comprising a first cap element (1), a second cap element (12), and a bridge (13) connecting said first element (1) and said second cap element (12), the first cap element (1) is configured for removable attachment to said first medicament holder receptacle (7a) and the second cap element (13) is configured for removable attachment to said second medicament holder receptacle (7b).

In one example, said base (7) comprises polypropylene, nylon, plastics, metals, or combinations thereof.

In one example, said first insert holder (8) comprises PTFE. In another example, said first holder insert holder comprises borosilicate glass.

In one example, said first cap liner (2) comprises PTFE, NXT 85 PTFE, fluoropolymer, silicone rubber, or plastics.

In one example, further comprising a layer of aluminum.

In one example, said second cap liner (11) comprises plastic, silicon rubber, paper, or combinations thereof.

In one example, said second medicament insert (9) comprises polypropylene. It will be appreciated that second insert holder (9) may comprise additional or alternative materials.

In one example, the first medicament holder (4) comprises PTFE, NTX 85 PTFE, glass, boroscilicate glass, brown borosilicate glass, Macor, or the like.

In one example, said first cap element (1) and said second cap element (12) comprise polypropylene, nylon, plastics, metals, or combinations thereof.

In one example, further comprising at least one first medicament (3) disposed within at least one aperture within said first medicament insert (4).

In one example, said at least one first medicament (3) is nitroglycerin (NTG).

In one example, further comprising three first medicaments (3) disposed in each of three apertures within said first medicament insert (4).

In one example, each said first medicament (3) comprises or consists of nitroglycerin (NTG).

In one example, said NTG comprises granules, a tablet(s), or oral fast dissolving film.

In one example, the oxygen within the aperture of the first medicament insert (4) is reduced by dilution with nitrogen gas or carbon dioxide.

In one example, further comprising at least one second medicament (10) disposed within at least one aperture with said second insert holder (9).

In one example, said second medicament (10) comprises or consists of acetylsalicylic acid (ASA).

In one example, further comprising four second medicaments (10) disposed in each of four aperture within said second medicament insert (9).

In one example, said second medicament (10) comprises or consists of acetylsalicylic acid (ASA).

In one example, each said ASA is 81 mg ASA.

In one example, further comprising instructions for use (7) on the surface of the base (5).

In one example, further comprising tamper-resistant tape placed the cap (14).

In one aspect there is described a method of storing nitroglycerin (NTG) and acetylsalicylic acid (ASA) for use in the treatment of known or suspect coronary artery disease (CAD), comprising: providing a medicament holder of any one of claims 1 to 21, said medicament holder comprising NTG and ASA.

In one aspect there is described a method of treating a human subject having or suspected of having coronary artery disease (CAD), comprising administering nitroglycerin (NTG) and acetylsalicylic acid (ASA) obtained from the medicament holder of any one of claims 1 to 21, said medicament holder comprising NTG and ASA.

In one aspect there is described a use of nitroglycerin (NTG) and acetylsalicylic acid (ASA) obtained from the medicament holder of any one of claims 1 to 21, said medicament holder comprising NTG and ASA for treating a human subject having or suspected of having coronary artery disease (CAD).

In one aspect there is described a kit for a medicament holder, comprising: a base (5) having a surface, the surface comprising a first medicament holder receptacle (5a) and a second medicament holder receptacle (5b); a first insert holder (8) removably disposed with the first medicament holder receptacle (5a); a second medicament insert (9) removably disposed with the second medicament holder (5b), the second medicament insert (9) is configured to removably store at least one second medicament (10); a first medicament insert (4) removably disposed with the first insert holder (8), the first medicament insert (4) is configured to removably store at least one first medicament (3); a first cap liner (2) removably disposed on the first insert holder (8); a second cap liner (11) removably disposed on the second insert holder (9); a cap (14) comprising a first cap element (1), a second cap element (12), and a bridge (13) connecting said first element (1) and said second cap element (12), the first cap element (1) is configured for removable attachment to said first medicament holder receptacle (7a) and the second cap element (13) is configured for removable attachment to said second medicament holder receptacle (7b).

In one example, said base (7) comprises polypropylene, nylon, plastics, metals, or combinations thereof.

In one example, said first holder insert holder (8) comprises PTFE. In another example, said first holder insert holder comprises borosilicate glass.

In one example, said first cap liner (2) comprises PTFE, NXT 85 PTFE, fluoropolymer, silicone rubber, or plastics.

In one example, further comprising a layer of aluminum.

In one example, said second cap liner (11) comprises plastic, silicon rubber, paper, or combinations thereof.

In one example, said second medicament insert (9) comprises polypropylene. It will be appreciated that second medicament insert (9) may comprise additional or alternative materials.

In one example, the first medicament holder (4) comprises PTFE, NTX 85 PTFE, glass, boroscilicate glass, brown borosilicate glass, Macor, or the like.

In one example, said first cap element (1) and said second cap element (12) comprise polypropylene, nylon, plastics, metals, or combinations thereof.

In one example, further comprising at least one first medicament (3) disposed within at least one aperture within said first medicament insert (4).

In one example, said at least one first medicament (3) is nitroglycerin (NTG).

In one example, further comprising three first medicaments (3) disposed in each of three apertures within said first medicament insert (4).

In one example, each said first medicament (3) comprises or consists of nitroglycerin (NTG).

In one example, said NTG comprises granules, a tablet(s), or oral fast dissolving film.

In one example, the oxygen within the aperture of the first medicament insert (4) is reduced by dilution with nitrogen gas or carbon dioxide.

In one example, further comprising at least one second medicament (10) disposed within at least one aperture with said second medicament insert (9).

In one example, said second medicament (10) comprises or consists of acetylsalicylic acid (ASA).

In one example, further comprising four second medicaments (10) disposed in each of four aperture within said second medicament insert (9).

In one example, said second medicament (10) comprises or consists of acetylsalicylic acid (ASA).

In one example, each said ASA is 81 mg ASA.

In one example, further comprising instructions for use (7) on the surface of the base (5).

In one example, further comprising tamper-resistant tape placed the cap (14).

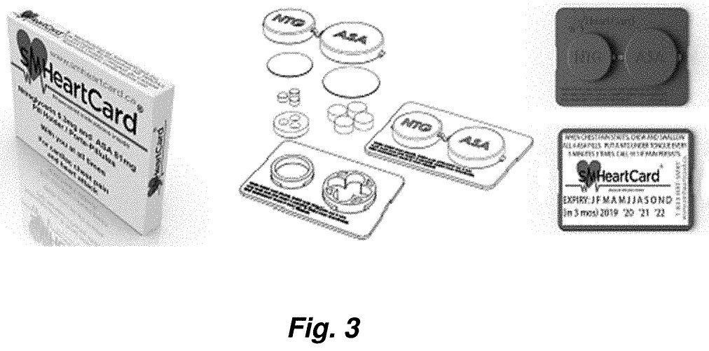

FIG. 3 depicts on example of an embodiment of the present application.

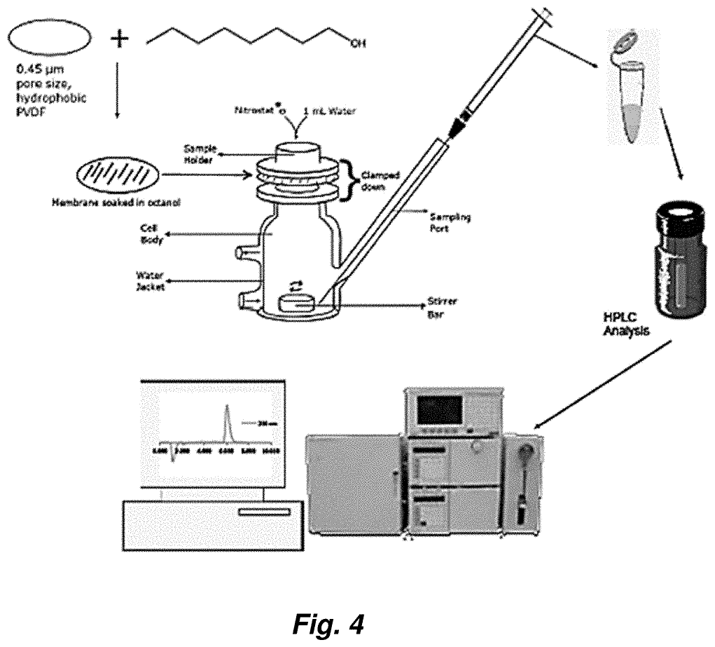

FIG. 4 depicts release test work flow.

FIG. 5 depicts Minimum and maximum pocket temperature measured with ThermPro-TP50 Digital LCD Indoor Thermometer Hygrometer Humidity Meter. On person carriage of the meter in shirt and pant pockets in winter and summer experienced temperature extremes ranging from 17.2° C. to 32.4° C.

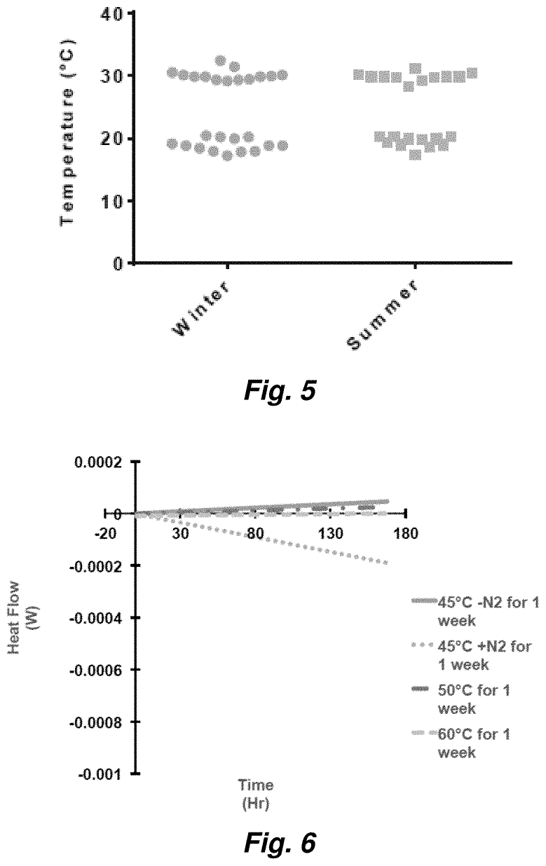

FIG. 6 depicts Heat flow of Nitrostat® at various temperatures for 1 week from the microcalorimeter. Nitrostat 6 was chemically stable for one week at 45° C. and 50° C., and N2 packaging did not improve stability. At 60° C. Nitrostat 6 demonstrated time dependent chemical changes.

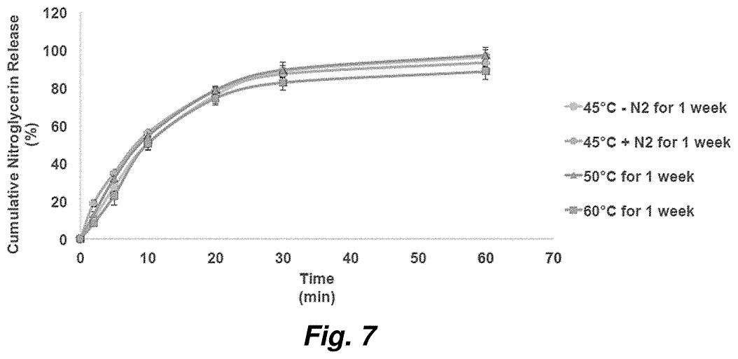

FIG. 7 depicts Release profiles of Nitrostat® at various temperatures from the microcalorimeter. (n=6 mean±SEM).

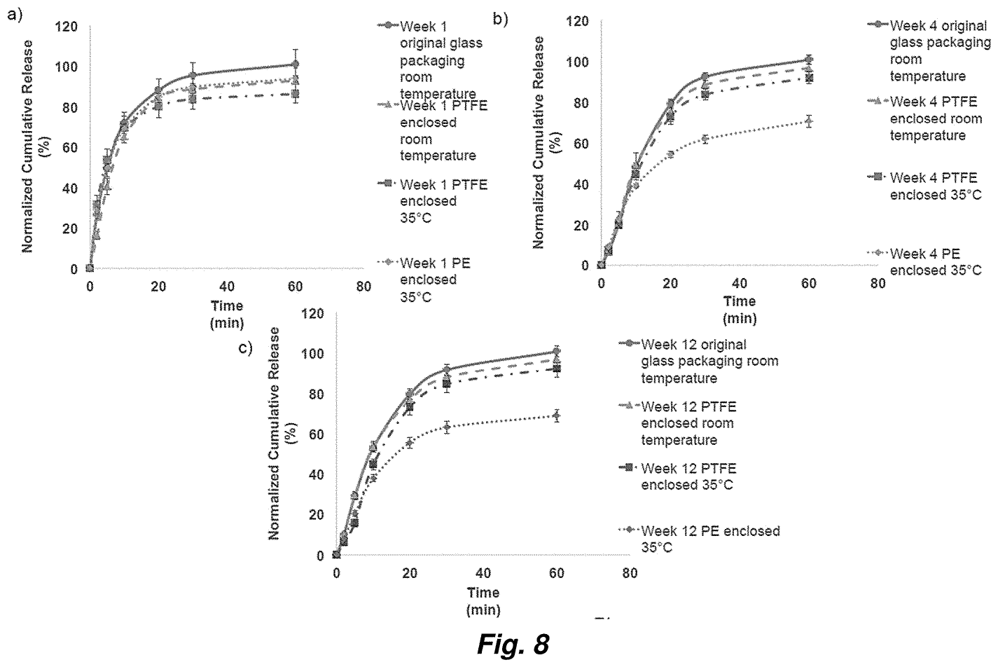

FIG. 8 depicts Release profiles of Nitrostat® in original glass packaging, emergency cardiac medication holder with NitroState enclosed with PTFE or PE at room temperature (22° C.), or 35° C. at week 1 (a), week 4 (b), and week 12 (c). (n=6 mean±SEM).

In one aspect there is described a method of packaging emergency cardiac mediations within a specially designed container system optimized to carry within a purse or billfold or wallet or pocket for immediate access and use with the onset of chest pain.

In one aspect there is described a method of packing solid pharmaceutical preparations containing the active substance glyceryl trinitrate/nitroglycerin (NTG), in the form of a single, double, or triple dose for oromucosal or oral administration, which is packaged in a base and well and insert and cap liner and cap system, wherein the well and cap liner are composed of materials facing the pharmaceutical preparation that have the chemical and physical properties to stabilize NTG on the surfaces facing the pharmaceutical preparation.

In one aspect there is described a method of packing solid pharmaceutical preparations containing the active substance NTG in a well and cap system, wherein the atmospheric content of Oxygen is reduced by dilution with Nitrogen gas, reducing oxidation of NTG.

In one aspect there is described a method of packing solid pharmaceutical preparations containing the active substance NTG in a well and cap system, wherein the atmospheric content of Oxygen is reduced by dilution with carbon dioxide gas, reducing oxidation of NTG.

In one aspect there is described a method of packing solid pharmaceutical preparations containing the active substance NTG in an opaque system, to reduce NTG degradation by light.

A method of packing solid pharmaceutical preparations containing the active substance NTG in a system that reduces NTG degradation triggered by temperature excursions outside the bounds of the manufacturer's directions.

In one aspect there is described preparation according to any one of the preceding where the NTG preparation is in the form of granules

In one aspect there is described preparation according to any one of the preceding where the NTG preparation is in the form of a tablet.

In one aspect there is described preparation according to any one of the preceding where the NTG preparation is in the form of an oral fast dissolving film.

In one aspect there is described a method of packaging four tablets of ASA 81 mg, and three tablets of sublingual/buccal NTG, for immediate access and administration at the onset of cardiac chest pain.

In one aspect there is described a method of packing solid pharmaceutical preparation containing the active substance ASA, in the form of a single, double, triple, or quadruple dose, for oral administration, wherein the pills are protected from excess humidity.

In one aspect there is described a method of packing ASA pills in a form that minimizes the depth of the required container.

In one aspect there is described a method of holding NTG pills in a custom well system holding individual NTG pills in an inert container with limited free volume to volatilize NTG gas and enhance the drug stability and dissolution properties.

In one aspect there is described a method of holding NTG pills in a custom well system holding individual NTG pills in an inert container with limited free volume to volatilize NTG gas and enhance the drug stability and dissolution properties, where that well is composed of borosilicate glass.

In one aspect there is described a method of holding NTG pills in a custom well system holding individual NTG pills in an inert container with limited free volume to volatilize NTG gas and enhance the drug stability and dissolution properties, where that well is composed of Macor.

In one aspect there is described method of holding NTG pills in a custom well system holding individual NTG pills in an inert container with limited free volume to volatilize NTG gas and enhance the drug stability and dissolution properties, where that well is composed of fluoropolymer plastic.

In one aspect there is described a method of holding NTG pills in a custom well system holding individual NTG pills in an inert container with limited free volume to volatilize NTG gas and enhance the drug stability and dissolution properties, where that well is composed of PTFE.

In one aspect there is described a method of holding NTG pills in a custom well system holding individual NTG pills in an inert container with limited free volume to volatilize NTG gas and enhance the drug stability and dissolution properties, where that well is composed of PTFE NXT 85.

In one aspect there is described a method of capping NTG pills in a custom well system holding individual NTG pills in an inert container with limited free volume to volatilize NTG gas, where the surface of the cap liner facing the NTG pill is composed of fluoropolymer plastic.

In one aspect there is described a method of capping NTG pills in a custom well system holding individual NTG pills in an inert container with limited free volume to volatilize NTG gas, where the surface of the cap liner facing the NTG pill is composed of PTFE.

In one aspect there is described a method of capping NTG pills in a custom well system holding individual NTG pills in an inert container with limited free volume to volatilize NTG, where the surface of the cap liner facing the NTG pill is composed of aluminum.

In one aspect there is described a method of capping NTG pills in a custom well system holding individual NTG pills in an inert container with limited free volume to volatilize NTG gas, where the surface facing the NTG pill is composed of fluoropolymer plastic backed by an impermeable barrier, such as aluminum foil.

In one aspect there is described a method of storing NTG pills that extends the stability and dissolution characteristics of NTG beyond the time to expiration interval recommended by the NTG pill manufacturer.

In one aspect there is described a cardiac medication holder with readily readable and simple instructions for emergency use of BOTH ASA and NTG.

In one aspect there is described a cardiac medication holder that simultaneously holds ASA and NTG.

In one aspect there is described a cardiac medication holder with readily identifiable location of both ASA and NTG for easy of access.

In one aspect there is described a cardiac medication holder designed to allow a tamper-resistant tape to cover the medication caps to ensure that the medications are undisturbed.

In one aspect there is described an apparatus designed to package cardiac medication for emergency use.

In one aspect there is described an apparatus designed to permit emergency cardiac medications to be carried in a pocket or in a wallet or in a purse for immediate access.

In one aspect there is described an apparatus assembled by performing the following steps in this sequence or variations thereof. The individual components of the cardiac medication holder are defined as shown in FIG. 1. The base is placed of a flat surface or a counter top. The NTG cap liner is placed inside the NTG cap. The NTG insert is placed into the NTG insert holder, and secured by glue or by friction fitting or by snapping into place. The ASA cap liner is placed into the ASA cap. The NTG cap is snapped onto the NTG insert holder. The ASA cap is snapped onto the ASA well.

In one aspect there is described an apparatus loaded with emergency cardiac medications as described here. The cardiac medication holder is placed flat on a counter top. The ASA cap is removed. Four 81 mg ASA are loaded into the ASA wells. The ASA cap is replaced. The NTG cap is removed. Three NTG pills are placed in the NTG wells. The NTG cap is replaced.

In one aspect there is described an apparatus that improves NTG stability by reducing atmospheric oxygen. To improve NTG stability, a syringe of inert gas (filled with either N2 or CO2), supplied in a kit or available to the person packaging the cardiac medication holder, is slowly injected into the NTG well system to dilute atmospheric oxygen, and then the NTG cap is replaced.

In one aspect there is described an apparatus that demonstrates secure sealing of the cardiac medication by means of applying tamper-resistance tape across the device. The tamper-resistant tape is placed across the device, leaving the instructions for use easily visible. The tamper-resistant tape fits into the groves to facilitate placement of the tape.

In one aspect there is described an apparatus that permits labeling to be readily attached to the bottom of the base. If prescribed by a physician and/or supplied by a pharmacist, the back of the device is suitable for adhesive labeling or written directions. This space could be used to record the expiry date of the medications, and contact information of the patient, and contact information of the patient's physician.

In one aspect there is described an apparatus to be used in the setting of cardiac or suspected cardiac chest pain. At the onset of cardiac chest pain or suspected cardiac chest pain, the user takes the device from his/her pocket, wallet, billfold, or purse, and places it on a flat surface. The ASA cap is removed. The four ASA 81 mg tablets are chewed and swallowed. The NTG cap is removed. The first NTG tablet is placed under the tongue and/or in contact with the oral mucosa. If chest pain persists after 5 minutes, the second NTG tablet is placed under the tongue and/or in contact with the oral mucosa. If chest pain persists after a further 5 minutes, the third NTG tablet is placed under the tongue and/or in contact with the oral mucosa. If chest pain persists, the instructions on the device recommend seeking emergency medical care.

ASSEMBLING THE CARDIAC MEDICATION HOLDER. Performing the following steps in this sequence or variations thereof assembles the cardiac medication holder. The individual components of the cardiac medication holder are defined as shown in FIG. 1. The base is placed of a flat surface or a counter top. The NTG cap liner is placed inside the NTG cap. The NTG insert is placed into the NTG insert holder, and secured by glue or by friction fitting or by snapping into place. The ASA cap liner is placed into the ASA cap. The NTG cap is snapped onto the NTG insert holder. The ASA cap is snapped onto the ASA well.

LOADING MEDICATION INTO THE CARDIAC MEDICATION HOLDER. The cardiac medication holder is placed flat on a counter top. The ASA cap is removed. Four 81 mg ASA are loaded into the ASA wells. The ASA cap is replaced. The NTG cap is removed. Three NTG pills are placed in the NTG wells. The NTG cap is replaced.

IMPROVING NTG STABILITY BY REDUCING ATMOSPHERIC OXYGEN. To improve NTG stability, a syringe of inert gas (filled with either N2 or CO2), supplied in a kit or available to the person packaging the cardiac medication holder, is slowly injected into the NTG well system to dilute atmospheric oxygen, and then the NTG cap is replaced.

DEMONSTRATING SECURE SEALING OF THE CARDIAC MEDICATION HOLDER. The tamper-resistant tape is placed across the device, leaving the instructions for use easily visible. The tamper-resistant tape fits into the groves (6) to facilitate placement of the tape.

LABELING. If prescribed by a physician and/or supplied by a pharmacist, the back of the device is suitable for adhesive labeling or written directions. This space could be used to record the expiry date of the medications, and contact information of the patient, and contact information of the patient's physician.

USE IN CARDIAC OR SUSPECTED CARDIAC CHEST PAIN. At the onset of cardiac chest pain or suspected cardiac chest pain, the user takes the device from his/her pocket, wallet, billfold, or purse, and places it on a flat surface. The ASA cap is removed. The four ASA 81 mg tablets are chewed and swallowed. The NTG cap is removed. The first NTG tablet is placed under the tongue and/or in contact with the oral mucosa. If chest pain persists after 5 minutes, the second NTG tablet is placed under the tongue and/or in contact with the oral mucosa. If chest pain persists after a further 5 minutes, the third NTG tablet is placed under the tongue and/or in contact with the oral mucosa. If chest pain persists, the instructions on the device recommend seeking emergency medical care.

### Laboratory Validation Studies

Background: Nitroglycerin is a potent vasodilatory drug used to alleviate chest pain. The American Heart Association Guidelines (O'Gara P T, Kushner F G, Ascheim D D, et al. 2013 ACCF/AHA Guideline for the Management of ST-Elevation Myocardial Infarction. J Am College Cardiol 2013;61:e78-140) recommend patients with coronary artery disease (CAD) to carry and use both aspirin (ASA) and nitroglycerin at the occurrence of pain. However, a recent Canadian survey revealed only 20% of patients with CAD carried the recommended medication (Horseman C, Frederick T. Poor compliance with carrying nitroglycerin and aspirin in patients with coronary artery disease. Can J Cardiol 2018;34:945.e1). A novel pill holder, described herein, was designed to provide a convenient way to immediately access nitroglycerin tablets and ASA in wallets, pockets, and purses. Hypothesis

Nitrostat® 0.3 mg pills stored within the cardiac emergency medication holder do not degrade significantly faster than those stored in their original borosilicate glass packaging container at room temperature.

### Temperature and Humidity Excursions with On-Body Carriage

Minimum and maximum pocket temperature measured with ThermPro-TP50 Digital LCD Indoor Thermometer Hygrometer Humidity Meter. Four individuals carried the meter in shirt or pants pockets continuously over 24 hours in both summer and winter conditions. On person carriage of the medicament holder in shirt and pant pockets in winter and summer experienced temperature extremes ranging from 17.2° C. to 32.4° C.

### Heat Flow

A microcalorimeter was used to measure the heat flow of the Nitrostat® pills in various temperatures to identify thermal chemical degradation. Nitrostat 6 was chemically stable for one week at 45° C. and 50° C., and N2 packaging did not improve stability. At 60° C. Nitrostat 6 demonstrated time dependent chemical changes.

### Storage Conditions

Nitrostat® pills were stored as in the original Pfizer packaging (control), emergency cardiac medication holder with inserts made from polytetrafluoroethylene (PTFE; a synthetic fluoropolymer), borosilicate glass, and polyethelene (PE). Cap liners were either PTFE coated or polyetheline. Temperatures tested were 4° C., room temperature (22° C.), and 35° C., stored with and without N2 gas to replace atmospheric oxygen. Pills were stored for 1, 2, 4, 8, 12 and 24 weeks before subjected to release tests to evaluate the performance. Additional studies were done at temperatures of −20° C. (data not shown; in preparation for publication).

### Release Test

A 0.45 μm synthetic hydrophobic PVDF membrane was briefly soaked in octanol to create a partition replicating a mucosal membrane. The membrane was assembled in a Franz cell with a Nitrostat® pill and 1 mL of water in the sample holder. The water jacket of the Franz cells were kept at 37.4° C. with the magnet stirrer spinning at 600 rpm. Samples were collected through a sampling port with a needle at 2, 5, 10, 20, 30, and 60 min.

### Analytical Method

The HPLC method was adapted from USP3 with some slight modifications. A C18 column was used with a mobile phase of methanol in water 45:55 on a Shimadzu HPLC with a UV detector at 210 nm. An injection volume of 50 μL was used with a flow rate of 0.75 mL/min.

### Statistical Analysis of Release Curves

F2 values comparing mean cumulative percent release of Nitroglycerin from the original glass packaging (OG) at room temperature (RT) and different test conditions. Glass=borosilicate glass enclosed. (n=6 mean±SEM).

Release curves demonstrated Nitrostat® pills performed similarly to original packaging at room temperature for all storage conditions for up to 12 weeks, except when enclosed in polyethylene. N2 gas packing made no difference in Nitrostat® performance. Furthermore, additional longer duration studies demonstrate Nitrostat® pills were stable when stored in PTFE enclosed conditions for up to 24 weeks, across temperatures ranging from 4° C. to 35° C. (data not show; in preparation for publication).

TABLE 1
F2 values comparing mean cumulative percent release of Nitroglycerin from the
original glass packaging (OG) at room temperature (RT) and different test conditions.

| | Similarity factor (f2) | | | | | | | | | |
|---|---|---|---|---|---|---|---|---|---|---|
| Comparision | Week 1 | | Week 2 | | Week 4 | | Week 8 | | Week 12 | |
| OG vs PTFE RT | 64.6 | SIMILAR | 69.4 | SIMILAR | 84.6 | SIMILAR | 86.6 | SIMILAR | 87.6 | SIMILAR |
| OG vs Glass RT | 46.9 | NOT | 59.8 | SIMLAR | 66.1 | SIMILAR | 69.5 | SIMILAR | 85.8 | SIMILAR |
| | | SIMILAR | | | | | | | | |
| OG vs PTFE 4° C. | 68.5 | SIMILAR | 76.1 | SIMILAR | 67.4 | SIMILAR | 68.9 | SIMILAR | 80.1 | SIMILAR |
| OG vs Glass 4° C. | 53.2 | SIMILAR | 59.6 | SIMILAR | 75.6 | SIMILAR | 85.0 | SIMILAR | 73.5 | SIMILAR |
| OG vs PTFE 35° C. | 62.3 | SIMILAR | 78.2 | SIMILAR | 71.1 | SIMILAR | 95.1 | SIMILAR | 64.8 | SIMILAR |
| OG vs PE 35° C. | 77.4 | SIMILAR | 82.3 | SIMILAR | 44.3 | NOT | 46.3 | NOT | 45.0 | NOT |
| | | | | | | SIMILAR | | SIMILAR | | SIMILAR |
| OG vs Glass 35° C. | 46.7 | NOT | 71.4 | SIMILAR | 64.1 | SIMILAR | 82.2 | SIMILAR | 58.1 | SIMILAR |
| | | SIMILAR | | | | | | | | |
| OG vs PTFE + N₂ | 58.8 | SIMILAR | 78.7 | SIMILAR | 65.1 | SIMILAR | 75.7 | SIMILAR | 84.2 | SIMILAR |
| 35° C. | | | | | | | | | | |
| OG vs Glass + N₂ | 55.0 | SIMILAR | 61.2 | SIMILAR | 75.8 | SIMILAR | 62.0 | SIMILAR | 77.1 | SIMILAR |
| 35° C. | | | | | | | | | | |

Glass = borosilicate glass enclosed.
PTFE = polytetrafluoroethylene enclosed.
PE = Polyethelene enclosed.
N2 = nitrogen gas packed.
(n = 6 mean ± SEM).

Nitrostat® pills performed similarly to original packaging at room temperature for all storage conditions, except when enclosed in PE. N2 packing made no difference in Nitrostat® performance.

### CONCLUSIONS

These laboratory validation studies demonstrate that throughout a 24 week testing period, Nitrostat® pills stored in enclosed in i) PTFE insert with PTFE cap liner, or ii) borosilicate glass insert with PTFE cap liner performed similarly to those stored in the original packaging across a range of temperatures relevant to on person carriage in a pocket or wallet. In contrast, enclosing Nitrostat® pills in Polyethylene significantly reduced nitroglycerin release. These results validate this emergency cardiac medication storage device for use at temperatures ranging from −20° C. to 35° C., and show that short (<1 week) exposure to temperatures up to 50° C. do not impair Nitrostat® performance.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A medicament holder, comprising:
a base (5) having a surface, the surface comprising a first medicament holder receptacle (5a) and a second medicament holder receptacle (5b);
a first insert holder (8) removably disposed with the first medicament holder receptacle (5a);
a second medicament insert (9) removably disposed with the second medicament holder (5b), the second medicament insert (9) is configured to removably store at least one second medicament (10);
a first medicament insert (4) removably disposed with the first insert holder (8), the first medicament insert (4) is configured to removably store at least one first medicament (3);
a first cap liner (2) removably disposed on the first insert holder (8);
a second cap liner (11) removably disposed on the second medicament insert (9); and
a cap (14) comprising a first cap element (1), a second cap element (12), and a bridge (13) connecting said first element (1) and said second cap element (12), the first cap element (1) is configured for removable attachment to said first medicament holder receptacle (7a) and the second cap element (13) is configured for removable attachment to said second medicament holder receptacle (7b).

2. The medicament holder of claim 1, wherein said base (7) comprises polypropylene, nylon, plastics, metals, or combinations thereof.

3. The medicament holder of claim 1 or 2, wherein said first holder insert holder (8) comprises PTFE

4. The medicament holder of any one of claims 1 to 3, wherein said first cap liner (2) comprises PTFE, NXT 85 PTFE, fluoropolymer, silicone rubber, or plastics.

5. The medicament holder of claim 4, further comprising a layer of aluminum.

6. The medicament holder of any one of claims 1 to 5, wherein said second cap liner (11) comprises plastic, silicon rubber, paper, or combinations thereof.

7. The medicament holder of any one of claims 1 to 6, wherein said second medicament holder (9) comprises polypropylene.

8. The medicament holder of any one of claims 1 to 7, wherein the first medicament holder (4) comprises PTFE, NTX 85 PTFE, glass, boroscilicate glass, brown borosilicate glass, Macor, or the like.

9. The medicament holder of any one of claims 1 to 8, wherein said first cap element (1) and said second cap element (12) comprise polypropylene, nylon, plastics, metals, or combinations thereof.

10. The medicament holder of any one claims 1 to 9, further comprising at least one first medicament (3) disposed within at least one aperture within said first medicament insert (4).

11. The medicament holder of claim 10, wherein said at least one first medicament (3) is nitroglycerin (NTG).

12. The medicament holder of any one of claims 1 to 9, further comprising three first medicaments (3) disposed in each of three apertures within said first medicament insert (4).

13. The medicament holder of claim 12, wherein each said first medicament (3) comprises or consists of nitroglycerin (NTG).

14. The medicament holder of claim 11 or 13, wherein said NTG comprises granules, a tablet(s), or oral fast dissolving film.

15. The medicament holder of any one of claims 1 to 14, wherein the oxygen within the aperture of the first medicament insert (4) is reduced by dilution with nitrogen gas or carbon dioxide.

16. The medicament holder of any one of claims 1 to 15, further comprising at least one second medicament (10) disposed within at least one aperture with said second insert holder (9).

17. The medicament holder of claim 16, wherein said second medicament (10) comprises or consists of acetylsalicylic acid (ASA).

18. The medicament holder of any one of claims 1 to 17, further comprising four second medicaments (10) disposed in each of four aperture within said second medicament holder (9).

19. The medicament holder of claim 18, wherein said second medicament (10) comprises or consists of acetylsalicylic acid (ASA).

20. The medicament holder of any one of claim 17 or 19, wherein each said ASA is 81 mg ASA.

21. The medicament holder of any one of claims 1 to 20, further comprising instructions for use (7) on the surface of the base (5).

22. The medicament holder of any one of claims 1 to 21, further comprising tamper-resistant tape placed the cap (14).

23. A method of storing nitroglycerin (NTG) and acetylsalicylic acid (ASA) for use in the treatment of known or suspect coronary artery disease (CAD), comprising: providing a medicament holder of any one of claims 1 to 22, said medicament holder comprising NTG and ASA.

24. A method of treating a human subject having or suspected of having coronary artery disease (CAD), comprising administering nitroglycerin (NTG) and acetylsalicylic acid (ASA) obtained from the medicament holder of any one of claims 1 to 22, said medicament holder comprising NTG and ASA.

25. Use of nitroglycerin (NTG) and acetylsalicylic acid (ASA) obtained from the medicament holder of any one of claims 1 to 22, said medicament holder comprising NTG and ASA for treating a human subject having or suspected of having coronary artery disease (CAD).

26. A kit for a medicament holder, comprising:
a base (5) having a surface, the surface comprising a first medicament holder receptacle (5a) and a second medicament holder receptacle (5b);
a first insert holder (8) removably disposed with the first medicament holder receptacle (5a);
a second medicament insert (9) removably disposed with the second medicament holder (5b), the second medicament insert (9) is configured to removably store at least one second medicament (10);
a first medicament insert (4) removably disposed with the first insert holder (8), the first medicament insert (4) is configured to removably store at least one first medicament (3);
a first cap liner (2) removably disposed on the first insert holder (8);
a second cap liner (11) removably disposed on the second insert holder (9);
a cap (14) comprising a first cap element (1), a second cap element (12), and a bridge (13) connecting said first element (1) and said second cap element (12), the first cap element (1) is configured for removable attachment to said first medicament holder receptacle (7a) and the second cap element (13) is configured for removable attachment to said second medicament holder receptacle (7b).

27. The kit of claim 26, wherein said base (7) comprises polypropylene, nylon, plastics, metals, or combinations thereof.

28. The kit of claim 26 or 27, wherein said first holder insert holder (8) comprises PTFE.

29. The kit of any one of claims 26 to 28, wherein said first cap liner (2) comprises PTFE, NXT 85 PTFE, fluoropolymer, silicone rubber, or plastics.

30. The kit of claim 29, further comprising a layer of aluminum.

31. The kit of any one of claims 26 to 30, wherein said second cap liner (11) comprises plastic, silicon rubber, paper, or combinations thereof.

32. The kit of any one of claims 26 to 31, wherein said second insert holder (9) comprises polypropylene.

33. The kit of any one of claims 26 to 32, wherein the first medicament holder (4) comprises PTFE, NTX 85 PTFE, glass, boroscilicate glass, brown borosilicate glass, Macor, or the like.

34. The kit of any one of claims 26 to 33, wherein said first cap element (1) and said second cap element (12) comprise polypropylene, nylon, plastics, metals, or combinations thereof.

35. The kit of any one claims 26 to 34, further comprising at least one first medicament (3) disposed within at least one aperture within said first medicament insert (4).

36. The kit of claim 35, wherein said at least one first medicament (3) is nitroglycerin (NTG).

37. The kit of any one of claims 36 to 34, further comprising three first medicaments (3) disposed in each of three apertures within said first medicament insert (4).

38. The kit of claim 37, wherein each said first medicament (3) comprises or consists of nitroglycerin (NTG).

39. The kit of claim 26 or 38, wherein said NTG comprises granules, a tablet(s), or oral fast dissolving film.

40. The kit of any one of claims 26 to 39, wherein the oxygen within the aperture of the first medicament insert (4) is reduced by dilution with nitrogen gas or carbon dioxide.

41. The kit of any one of claims 26 to 40, further comprising at least one second medicament (10) disposed within at least one aperture with said second insert holder (9).

42. The kit of claim 41, wherein said second medicament (10) comprises or consists of acetylsalicylic acid (ASA).

43. The kit of any one of claims 26 to 40, further comprising four second medicaments (10) disposed in each of four aperture within said second medicament insert (9).

44. The kit of claim 43, wherein said second medicament (10) comprises or consists of acetylsalicylic acid (ASA).

45. The kit of any one of claim 42 or 44, wherein each said ASA is 81 mg ASA.

46. The kit of any one of claims 26 to 45, further comprising instructions for use (7) on the surface of the base (5).

47. The kit of any one of claims 26 to 46, further comprising tamper-resistant tape placed the cap (14).
